(19) United States
(12) Patent Application Publication
(10) Pub. No.: US 2007/0141211 A1
(43) Pub. Date: Jun. 21, 2007

(54) Encapsulated Phospholipid-Stabilized Oxidizable Material

(75) Inventors: Charles W. Kolar JR., St. Louis, MO (US); Joshua J. Moore, Belleville, IL (US)

Correspondence Address: SOLAE, LLC, P. O. BOX 88940, ST. LOUIS, MO, 63188, US

(73) Assignee: Solae, LLC, St. Louis, MO (US)

(21) Appl. No.: 11/610,885

(22) Filed: Dec. 14, 2006

Related U.S. Application Data
(60) Provisional application No. 60/751,020, filed on Dec. 16, 2005.

(51) Int. Cl. A61K 9/28; B05D 1/36; A23L 1/00; B06B 1/20

(52) U.S. Cl. 426/302; 427/601

(57) ABSTRACT

Compositions and methods to reduce the oxidation of an oxidizable material are disclosed herein. The invention provides a microcapsule comprising a core material, which is the phospholipid-stabilized oxidizable material, and a shell wall that encapsulates the core material. Food products comprising an edible material and a microcapsule of phospholipid-stabilized oxidizable material are also disclosed.

### CROSS REFERENCE TO RELATED APPLICATIONS

The application claims priority from Provisional Application Ser. No. 60/751,020 filed on Dec. 16, 2005, which is hereby incorporated by reference in its entirety.

### FIELD OF THE INVENTION

The present invention provides encapsulated compositions and methods for reducing the oxidation of an oxidizable material in a substantially water-free environment.

### BACKGROUND OF THE INVENTION

Consumption of foods rich in omega-3 polyunsaturated fatty acids (PUFAs) has been associated with decreased cardiovascular death by decreasing plasma triglycerides, blood pressure, platelet aggregation, and inflammation. While seafood is the best source of omega-3 acids, many individuals do not like the taste of seafood, do not have ready access to seafood, or cannot afford seafood. One solution is to supplement the diet with cod liver oil or fish oil capsules, but this solution has low compliance. Another solution is to add omega-3 rich fish oils directly to foods, such as dairy products, cereal products, baked goods, and nutrition bars.

A challenge with the latter approach is to provide the benefits of omega-3 fatty acids without imparting any offending fish flavors or fish odors, which are byproducts of lipid oxidation. A need exists, therefore, for a stabilized preparation of PUFAs that can be added to low moisture or high moisture foods, such that the PUFAs are protected from oxidation.

### SUMMARY OF THE INVENTION

One aspect of the present invention provides a microcapsule comprising a core material and a shell wall that encapsulates the core material. The core material comprises an oxidizable material and a phospholipid, wherein the concentration of the phospholipid in the core material is from about 2% to about 50% by weight of the oxidizable material.

Another aspect of the invention encompasses a food product comprising an edible material and a microcapsule. The microcapsule comprises a core material and a shell wall encapsulating the core material. The core material comprises an oxidizable material and a phospholipid, wherein the concentration of the phospholipid in the core material is from about 2% to about 50% by weight of the oxidizable material.

A still further aspect of the invention provides a method for reducing the oxidation of an oxidizable material. The method comprises contacting the oxidizable material with a phospholipid in a substantially water-free environment, wherein the percentage of phospholipid is from about 2% to about 50% by weight of the oxidizable material.

Other aspects and features of the invention are described in more detail below.

### DESCRIPTION OF THE FIGURES

FIG. 1 illustrates that microcapsules have greater oxidative stability than lecithin-stabilized oils containing the same percentage of lecithin. The stability of both preparations was measured using the oxidative stability index (OSI) method. OSI values (in hours) are plotted as a function of the percentage of lecithin in the different preparations.

FIG. 2 illustrates that a lecithin-stabilized oil comprising 20% lecithin has the lowest levels of peroxides. The level of peroxides was measured in a lecithin-stabilized oils comprising from 3.1% to 40% lecithin at several time points over 24 days. The peroxide values (PV) are plotted for each lecithin-stabilized oil as a function of time.

FIG. 3 illustrates that lecithin-stabilized oils comprising about 25-30% lecithin have the lowest peroxide values. Shown is a quadratic term plot in which PV values are plotted as a function of lecithin percentage and time.

FIG. 4 illustrates the development of propanal over time in microcapsules comprising 12% lecithin. The areas under the peaks from the GC plots are plotted versus time.

FIG. 5 illustrates the development of hexanal over time in microcapsules comprising 12% lecithin. The areas under the peaks from the GC plots are plotted versus time.

FIG. 6 presents a TEM image of a microcapsule comprising 6.4% lecithin (and soy protein).

FIG. 7 illustrates that additional antioxidants provide increased oxidative stability to microcapsules comprising 6.4% or 30% lecithin. The OSI values are plotted for each type of microcapsule.

FIG. 8 presents the levels of specific volatiles in different preparations lecithin-stabilized oils prepared with or without additional antioxidants.

FIG. 9 illustrates that microcapsules comprising about 20% lecithin have the lowest level of fish flavor as determined by a sensory quality system, the Solae Qualitative Screening (SQS) method. The mean fishy scores are plotted as a function of lecithin percentage.

FIG. 10 illustrates that chocolate flavored bars with microcapsules comprising 30% lecithin have a better sensory profile than chocolate flavored bars with microcapsules comprising 6.4% lecithin. Directional differences from the control are plotted for each attribute for each bar.

### DETAILED DESCRIPTION OF THE INVENTION

The present invention provides compositions and methods to reduce the oxidation of an oxidizable material. In particular, the invention provides a microcapsule comprising a core of phospholipid-stabilized oxidizable material that is surrounded by a shell wall. It has been discovered, as demonstrated in the examples, that contact of an oxidizable material, such as an omega-3 fatty acid, with a phospholipid, such as lecithin (at about 2% to about 50% by weight of the oxidizable material) dramatically reduces the oxidation of the oxidizable material that is substantially water-free. This key discovery provides means to include omega-3 fatty acids or other oxidizable materials in foods without imparting offensive tastes or odors to the foods from the oxidation of the fatty acids or other oxidizable materials.

(I) Composition

One aspect of the invention is a composition comprising an oxidizable material and a phospholipid, wherein the concentration of the phospholipid in the composition is from about 2% to about 50% by weight of the oxidizable material. In an exemplary embodiment, the concentration of the phospholipid in the composition is from about 25% to about 30% by weight of the oxidizable material. The phospholipid reduces the oxidation of the oxidizable material. To make the composition, the phospholipid is contacted with a solvent and an oxidizable material to form a mixture, and then the solvent is removed from the mixture to form the phospholipid-stabilized oxidizable material. Suitable oxidizable materials and phospholipids are described below.

(a) Oxidizable Material

An oxidizable material having utility in the present invention includes a material comprising a molecule with a carbon backbone having at least one carbon-carbon double bond that is prone to oxidation. Removal of a labile hydrogen atom from a carbon adjacent to the double bond creates a free radical that is susceptible to attack by oxygen to form a free radical peroxide, which may serve as a catalyst for further oxidation. The oxidation of the oxidizable material may be determined using the oxygen stabilization method (OSI) or the peroxide value (PV) method, as detailed in the examples.

A variety of oxidizable materials are suitable for use in this invention. In general, the oxidizable material comprises at least one oxidizable lipid. Oxidizable lipids include fatty acids, fatty acid esters, fatty acid methyl esters (FAMEs), glycerides, glycolipids, phospholipids, sphingolipids, cholesterol, steroid hormones, sterols, and polyisoprenoids.

In one embodiment, the oxidizable material may be derived from a biological source, such that it may be a crude mixture of proteins, lipids, and carbohydrates. In another embodiment, the oxidizable material may be a mixture of lipids that is essentially devoid of proteins and/or carbohydrates. In yet another embodiment, the oxidizable material may be a purified lipid.

In still another embodiment, the oxidizable material may be a preparation of substantially unsaturated fats or substantially unsaturated oils. In general, fats and oils comprise monoglycerides, diglycerides, triglycerides, and free fatty acids. The glycerides of fats and oils generally comprise fatty acids that are at least 4 carbons in length, and more preferably, unsaturated fatty acids that range in length from 16 to 24 carbons. The unsaturated fatty acid may be monounsaturated or polyunsaturated.

In another embodiment, the oxidizable material may be a polyunsaturated fatty acid (PUFA), which has at least two carbon-carbon double bonds generally in the cis-configuration. The PUFA may be a long chain fatty acid having at least 18 carbons atoms. The PUFA may be an omega-3 fatty acid in which the first double bond occurs in the third carbon-carbon bond from the methyl end of the carbon chain (i.e., opposite the carboxyl acid group). Examples of omega-3 fatty acids include alpha-linolenic acid (18:3, ALA), stearidonic acid (18:4), eicosatetraenoic acid (20:4), eicosapentaenoic acid (20:5; EPA), docosatetraenoic acid (22:4), n-3 docosapentaenoic acid (22:5; n-3DPA), and docosahexaenoic acid (22:6; DHA). The PUFA may also be an omega-6 fatty acid, in which the first double bond occurs in the sixth carbon-carbon bond from the methyl end. Examples of omega-6 fatty acids include linoleic acid (18:2), gamma-linolenic acid (18:3), eicosadienoic acid (20:2), dihomo-gamma-linolenic acid (20:3), arachidonic acid (20:4), docosadienoic acid (22:2), adrenic acid (22:4), and n-6 docosapentaenoic acid (22:5). The fatty acid may also be an omega-9 fatty acid, such as oleic acid (18:1), eicosenoic acid (20:1), mead acid (20:3), erucic acid (22:1), and nervonic acid (24:1).

In another embodiment, the oxidizable material may be a seafood-derived oil. The seafood may be a vertebrate fish or a marine organism, such that the oil may be a fish oil or a marine oil. The long chain (20C, 22C) omega-3 and omega-6 fatty acids are found in seafood. The ratio of omega-3 to omega-6 fatty acids in seafood ranges from about 8:1 to 20:1. Seafood from which oil rich in omega-3 fatty acids may be derived include, but are not limited to, abalone scallops, albacore tuna, anchovies, catfish, clams, cod, gem fish, herring, lake trout, mackerel, menhaden, orange roughy, salmon, sardines, sea mullet, sea perch, shark, shrimp, squid, trout, and tuna.

In yet another embodiment, the oxidizable material may be a plant-derived oil. Plant and vegetable oils are rich in omega-6 fatty acids. Some plant-derived oils, such as flaxseed oil, are especially rich in omega-3 fatty acids. Plant or vegetable oils are generally extracted from the seeds of a plant, but may also be extracted from other parts of the plant. Plant or vegetable oils that are commonly used for cooking or flavoring include, but are not limited to, acai oil, almond oil, amaranth oil, apricot seed oil, argan oil, avocado seed oil, babassu oil, ben oil, blackcurrant seed oil, Borneo tallow nut oil, borage seed oil, buffalo gourd oil, canola oil, carob pod oil, cashew oil, castor oil, coconut oil, coriander seed oil, corn oil, cottonseed oil, evening primrose oil, false flax oil, flax seed oil, grapeseed oil, hazelnut oil, hemp seed oil, kapok seed oil, lallemantia oil, linseed oil, macadamia oil, meadowfoam seed oil, mustard seed oil, okra seed oil, olive oil, palm oil, palm kernel oil, peanut oil, pecan oil, pequi oil, perilla seed oil, pine nut oil, pistachio oil, poppy seed oil, prune kernel oil, pumpkin seed oil, quinoa oil, ramtil oil, rice bran oil, safflower oil, sesame oil, soybean oil, sunflower oil, tea oil, thistle oil, walnut oil, or wheat germ oil. The plant derived oil may also be hydrogenated or partially hydrogenated.

In still a further embodiment, the oxidizable material may be an algae-derived oil. Commercially available algae-derived oils include those from Crypthecodinium cohnii and Schizochytrium sp. Other suitable species of algae, from which oil is extracted, include Aphanizomenon flos-aquae, Bacilliarophy sp., Botryococcus braunii, Chlorophyceae sp., Dunaliella tertiolecta, Euglena gracilis, Isochrysis galbana, Nannochloropsis salina, Nannochloris sp., Neochloris oleoabundans, Phaeodactylum tricornutum, Pleurochrysis carterae, Prymnesiumparvum, Scenedesmus dimorphus, Spirulina sp., and Tetraselmis chui.

In an alternate embodiment, the oxidizable material may be a spice or fragrance oil. Suitable examples of spice or fragrant oils include angelica oil, anise oil, basil oil, bergamont oil, orange oil, black pepper oil, calamus oil, citronella oil, calendula oil, camphor oil, cardamom oil, celery oil, chamomile oil, cinnamon oil, clove oil, coriander oil, lemon grass oil, cypress oil, cumin seed oil, davana oil, dill seed oil, eucalyptus oil, fennel seed oil, garlic oil, geranium oil, ginger oil, grape seed oil, hyssop oil, jasmine oil, juniper berry oil, lavender oil, lemon oil, lime oil, myrrh oil, neroli oil, neem oil, nutmeg oil, palm Rosa oil, parsley oil, peppermint oil, rose oil, rosemary oil, rose wood oil, sage oil, sesame oil, spearmint oil, tarragon oil, tea tree oil, thyme oil, tangerine oil, turmeric root oil, vetiver oil, wormwood oil, and yara yara oil.

In yet another embodiment, the oxidizable material may be a pharmaceutical formulation comprising an oxidatively unstable pharmaceutical, such as arachadonic acid or a prostaglandin. The formulation may also comprise an unstable oil as a carrier. Suitable examples of pharmaceutical grade carrier oils include cod liver oil, corn oil, cottonseed oil, eucalyptus oil, lavender oil, olive oil, peanut oil, peppermint oil, safflower oil, sesame oil, and soybean oil. The oxidizable material may also be a formulation comprising a fat-soluble vitamin, such as vitamin A, D, K, or E.

In an alternate embodiment, the oxidizable material may be preparation of fish materials or fish meal, which is the solid material that remains after most of the water and oil have been removed from the starting fish material. Non-limiting examples of fish or marine organism that may be used for the preparation of fish meal include anchovy, blue whiting, capelin, crab, herring, mackerel, menhaden, pollack, salmon, shrimp, squid, tuna, and whitefish.

In still another embodiment, the oxidizable material may be an animal-derived fat. Non-limiting examples of suitable animal-derived fats include poultry fat, beef tallow, mutton tallow, butter, pork lard, whale blubber, and yellow grease (which may be a mixture of vegetable and animal fats).

In a preferred embodiment, the oxidizable material is seafood oil comprising omega-3 and omega-6 fatty acids. In another preferred embodiment, the oxidizable material is an omega-3 fish oil. In yet another preferred embodiment, the oxidizable material is an omega-3 fatty acid.

(b) Phospholipid

The composition further comprises a phospholipid to stabilize the oxidizable material and thus, to reduce its oxidation. A phospholipid comprises a backbone, a negatively charged phosphate group attached to an alcohol, and at least one fatty acid. Phospholipids having a glycerol backbone comprise two fatty acids and are termed glycerophospholipids. Examples of a glycerophospholipid include phosphatidylcholine, phosphatidylethanolamine, phosphatidylinositol, phosphatidylserine, and diphosphatidylglycerol (i.e., cardiolipin). Phospholipids having a sphingosine backbone are called sphingomyelins. The fatty acids attached via ester bonds to the backbone of a phospholipid tend to be 12 to 22 carbons in length, and some may be unsaturated. For example, phospholipids may contain oleic acid (18:1), linoleic acid (18:2, an omega-6), and alpha-linolenic acid (18:3, an omega-3). The two fatty acids of a phospholipid may be the same or they may be different; e.g., dipalmitoylphosphatidylcholine, 1-stearyoyl-2-myristoylphosphatidylcholine, or 1-palmitoyl-2-linoleoylethanolamine.

In one embodiment, the phospholipid may be a single purified phospholipid, such as distearoylphosphatidylcholine. In another embodiment, the phospholipid may be mixture of purified phospholipids, such as a mix of phosphatidylcholines. In still another embodiment, the phospholipid may be a mixture of different types of purified phospholipids, such as a mix of phosphatidylcholines and phosphatidylinositols or a mixture of phosphatidylcholines and phosphatidylethanolamines.

In an alternate embodiment, the phospholipid may be a complex mix of phospholipids, such as a lecithin. Lecithin is found in nearly every living organism. Commercial sources of lecithin include soybeans, rice, sunflower seeds, chicken egg yolks, milk fat, bovine brain, bovine heart, and algae. In its crude form, lecithin is a complex mixture of phospholipids, glycolipids, triglycerides, sterols and small quantities of fatty acids, carbohydrates and sphingolipids. Soy lecithin is rich in phosphatidylcholine, phosphatidylethanolamine, phosphatidylinositol, and phosphatidic acid. Lecithin may be de-oiled and treated such that it is an essentially pure mixture of phospholipids. Lecithin may be modified to make the phospholipids more water-soluble. Modifications include hydroxylation, acetylation, and enzyme treatment, in which one of the fatty acids is removed by a phospholipase enzyme and replaced with a hydroxyl group.

In yet an alternate embodiment, the phospholipid may be a soy lecithin produced under the trade name Solec by the Solae Company (St. Louis, Mo.). The soy lecithin may be Solec®F, a dry, de-oiled, non enzyme modified preparation containing about 97% phospholipids. The soy lecithin may be Solec®8160, a dry, de-oiled, enzyme modified preparation containing about 97% phospholipids. The soy lecithin may be Solec®8120, a dry, de-oiled, hydroxylated preparation containing about 97% phospholipids. The soy lecithin may be Solec®8140, a dry, de-oiled, heat resistant preparation containing about 97% phospholipids. The soy lecithin may be Solec®R, a dry, de-oiled preparation in granular form containing about 97% phospholipids.

In a preferred embodiment, the phospholipid is phosphatidylcholine. In another preferred embodiment, the phospholipid is phosphatidylethanolamine. In an especially preferred embodiment the phospholipid is lecithin. In an exemplary embodiment, the phospholipid is soy lecithin.

The ratio of the phospholipid to the oxidizable material can and will vary depending upon the nature of the oxidizable material and the phospholipid preparation. In particular, the concentration of phospholipid will be of a sufficient amount to prevent the oxidation of the oxidizable material. The concentration of the phospholipid will generally range from about 1% to about 65% by weight of the oxidizable material. In one embodiment, the concentration of the phospholipid may range from about 2% to about 50% by weight of the oxidizable material. In another embodiment, the concentration of the phospholipid may range from about 2% to about 10% by weight of the oxidizable material. In an alternate embodiment, the concentration of the phospholipid may range from about 10% to about 20% by weight of the oxidizable material. In yet another embodiment, the concentration of the phospholipid may range from about 20% to about 30% by weight of the oxidizable material. In still another embodiment, the concentration of the phospholipid may range from about 30% to about 40% by weight of the oxidizable material. In another alternate embodiment, the concentration of the phospholipid may range from about 40% to about 50% by weight of the oxidizable material. In a preferred embodiment, the concentration of the phospholipid may range from about 15% to about 35% by weight of the oxidizable material. In an especially exemplary embodiment, concentration of the phospholipid may range from about 25% to about 30% by weight of the oxidizable material.

The type of oxidizable material and the type of phospholipid comprising the composition can and will vary depending upon the intended application or use of the composition. Table A presents non-limiting examples of oxidizable materials and phospholipids that may be combined in the composition of the invention.

TABLE A
Compositions of the invention.

| Oxidizable Material | Phospholipid |
|---|---|
| Biological sample | soy lecithin |
| Biological sample | egg yolk lecithin |
| Biological sample | milk lecithin |
| Biological sample | rice lecithin |
| Biological sample | purified soy lecithin (PC, PE, PI, PA) |
| Biological sample | phosphatidylcholine (PC) |
| Biological sample | phosphatidylethanolamine (PE) |
| Biological sample | phosphatidylinositol (PI) |
| Biological sample | phosphatidic acid (PA) |
| Biological sample | phosphatidylserine |
| Biological sample | diphosphatidyl glycerol |
| Unsaturated fats or oils | soy lecithin |
| Unsaturated fats or oils | egg yolk lecithin |
| Unsaturated fats or oils | milk lecithin |
| Unsaturated fats or oils | rice lecithin |
| Unsaturated fats or oils | purified soy lecithin (PC, PE, PI, PA) |
| Unsaturated fats or oils | phosphatidylcholine (PC) |
| Unsaturated fats or oils | phosphatidylethanolamine (PE) |
| Unsaturated fats or oils | phosphatidylinositol (PI) |
| Unsaturated fats or oils | phosphatidic acid (PA) |
| Unsaturated fats or oils | phosphatidylserine |
| Unsaturated fats or oils | diphosphatidyl glycerol |
| Fish oil | soy lecithin |
| Fish oil | egg yolk lecithin |
| Fish oil | milk lecithin |
| Fish oil | rice lecithin |
| Fish oil | purified soy lecithin (PC, PE, PI, PA) |
| Fish oil | phosphatidylcholine (PC) |
| Fish oil | phosphatidylethanolamine (PE) |
| Fish oil | phosphatidylinositol (PI) |
| Fish oil | phosphatidic acid (PA) |
| Fish oil | phosphatidylserine |
| Fish oil | diphosphatidyl glycerol |
| Marine oil | soy lecithin |
| Marine oil | egg yolk lecithin |
| Marine oil | milk lecithin |
| Marine oil | rice lecithin |
| Marine oil | purified soy lecithin (PC, PE, PI, PA) |
| Marine oil | phosphatidylcholine (PC) |
| Marine oil | phosphatidylethanolamine (PE) |
| Marine oil | phosphatidylinositol (PI) |
| Marine oil | phosphatidic acid (PA) |
| Marine oil | phosphatidylserine |
| Marine oil | diphosphatidyl glycerol |
| Vegetable oil | soy lecithin |
| Vegetable oil | egg yolk lecithin |
| Vegetable oil | milk lecithin |
| Vegetable oil | rice lecithin |
| Vegetable oil | purified soy lecithin (PC, PE, PI, PA) |
| Vegetable oil | phosphatidylcholine (PC) |
| Vegetable oil | phosphatidylethanolamine (PE) |
| Vegetable oil | phosphatidylinositol (PI) |
| Vegetable oil | phosphatidic acid (PA) |
| Vegetable oil | phosphatidylserine |
| Vegetable oil | diphosphatidyl glycerol |
| Algal oil | soy lecithin |
| Algal oil | egg yolk lecithin |
| Algal oil | milk lecithin |
| Algal oil | rice lecithin |
| Algal oil | purified soy lecithin (PC, PE, PI, PA) |
| Algal oil | phosphatidylcholine (PC) |
| Algal oil | phosphatidylethanolamine (PE) |
| Algal oil | phosphatidylinositol (PI) |
| Algal oil | phosphatidic acid (PA) |
| Algal oil | phosphatidylserine |
| Algal oil | diphosphatidyl glycerol |
| Omega-3 fatty acid | soy lecithin |
| Omega-3 fatty acid | egg yolk lecithin |
| Omega-3 fatty acid | milk lecithin |
| Omega-3 fatty acid | rice lecithin |
| Omega-3 fatty acid | purified soy lecithin (PC, PE, PI, PA) |
| Omega-3 fatty acid | phosphatidylcholine (PC) |
| Omega-3 fatty acid | phosphatidylethanolamine (PE) |
| Omega-3 fatty acid | phosphatidylinositol (PI) |
| Omega-3 fatty acid | phosphatidic acid (PA) |
| Omega-3 fatty acid | phosphatidylserine |
| Omega-3 fatty acid | diphosphatidyl glycerol |

In an exemplary embodiment, the phospholipid is a lecithin, and the oxidizable material is a seafood oil comprising omega-3 and omega-6 fatty acids. In an alternative exemplary embodiment, the phospholipid is a lecithin, and the oxidizable material is an omega-3 fatty acid. In each of these embodiments, the concentration of the lecithin in the composition is from about 2% to about 50% by weight of the oxidizable material, and more typically, from about 15% to about 35% by weight of the oxidizable material. In an exemplary embodiment, the concentration of the lecithin in the composition is from about 25% to about 30% by weight of the oxidizable material.

(c) Additional Components

The composition may further comprise at least one protein. The protein may be a vegetable protein, an animal protein, a fungal protein, a microbial protein, or a mixture thereof. Non-limiting examples of an animal protein suitable for use in this invention include casein, dairy whey protein, gelatin, or a mixture thereof. Non-limiting examples of a vegetable protein include soy protein, corn protein, wheat protein, rice protein, canola protein, pea protein, or a mixture thereof. The corn protein may be corn gluten meal, or more preferably, zein. The wheat protein may be wheat gluten. A preferred vegetable protein is soy protein.

The soy protein may be provided by a preparation of soy flour, soy protein concentrate, or soy protein isolate. These preparations of soy protein are typically formed from a soybean starting material, which may be soybeans or a soybean derivative. Preferably, the soybean starting material may be soybean cake, soybean chips, soybean meal, soybean flakes, or a mixture of these materials. The soybean cake, chips, meal, or flakes may be formed from soybeans according to conventional procedures in the art. That is, soybean cake and soybean chips are generally formed by extraction of part of the oil from soybeans by pressure or solvents; soybean flakes are generally formed by cracking, heating, and flaking soybeans and reducing the oil content of the soybeans by solvent extraction; and soybean meal is generally formed by grinding soybean cake, chips, or flakes.

The protein may be modified using procedures known in the art to improve the utility or characteristics of the protein. The modifications include, but are not limited to, denaturation or hydrolysis of the protein. The denaturation or hydrolysis may be chemically mediated or it may be enzymatic.

The composition may further comprise at least one additional antioxidant that is not a phospholipid or a lecithin. The additional antioxidant may further stabilize the oxidizable material. The antioxidant may be natural or synthetic. Suitable antioxidants include, but are not limited to, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, camosol, carvacrol, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rice bran extract, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof. Preferred antioxidants include tocopherols, ascorbyl palmitate, and rosemary extract. The concentration of the additional antioxidant or combination of antioxidants may range from about 0.001% to about 5% by weight, and preferably from about 0.01% to about 1% by weight.

(d) Forming the Composition

The composition of the invention, i.e., the phospholipid-stabilized oxidizable material, is generally formed by first contacting the phospholipid with a solvent. The solvent may be polar or non-polar. Non-limiting examples of polar solvents include water, ethanol, glycerol, propylene glycol, or combinations thereof. Non-limiting examples of a non-polar solvent include pentane, hexane, heptane, or petroleum ether (which is a mixture of pentane, hexane, and heptane). The mixture of phospholipid and solvent may be heated, stirred, and/or mixed by homogenization. An oxidizable material is then contacted with the mixture of phospholipid and solvent, and again the mixture may be heated, stirred, and/or mixed by homogenization. In some embodiments, at least one protein or at least one additional antioxidant may be added to the mixture.

In embodiments comprising a polar solvent, an emulsion may be formed comprising droplets of phospholipid and oxidizable material in the aqueous solvent. The droplets in the emulsion may be encapsulated using methods described in section (II)(d). Alternatively, the aqueous phase may be removed from the emulsion by techniques well known in the art, such as spray drying, freeze drying, or vacuum evaporation. The resultant phospholipid-stabilized oxidizable material is stable, provided it remains substantially water-free. The phospholipid-stabilized oxidizable material may also be encapsulated by methods described in section (II)(d).

In embodiments comprising a non-polar solvent, a homogeneous mixture is generally formed. The non-polar solvent may be removed from the mixture to form the phospholipid-stabilized oxidizable material. Alternatively, microcapsules comprising the phospholipid-stabilized oxidizable material may be formed from the mixture using a method described in section (II)(d). The solvent may be removed before or during the encapsulation process.

(II) Microcapsule

To provide a substantially water-free environment for the composition of the invention, another aspect of the invention provides a microcapsule comprising a core material and a shell wall that encapsulates the core material. The core material comprises phospholipid-stabilized oxidizable material, wherein the concentration of the phospholipid ranges from about 2% to about 50% by weight of the oxidizable material. The shell wall protects the core material such that it is in a substantially water-free environment.

(a) Core Material

The core material of the microcapsule comprises an oxidizable material as described in section (I)(a) and a phospholipid as described in section (I)(b) that were combined to form the phospholipid-stabilized oxidizable material as described in section (I)(d). The core material may further comprise at least one protein or at least one additional antioxidant that is not a phospholipid or a lecithin, as described in section (I)(c).

(b) Shell Wall

As will be appreciated by a skilled artisan, the materials that comprise the shell wall can and will vary depending upon a variety of factors, including, the core material, and the intended use of the microcapsule. Generally speaking, if the microcapsule is to be utilized in a food application, preferably the shell wall is food grade material. The shell wall material may be a biopolymer, a semi-synthetic polymer, or a mixture thereof. The microcapsule may comprise one shell wall layer or many shell wall layers, of which the layers may be of the same material or different materials.

In one embodiment, the shell wall material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum.

In another embodiment, the shell wall material may comprise a protein. Suitable proteins include, but are not limited to, gelatin, casein, collagen, whey proteins, soy proteins, rice protein, and corn proteins.

In an alternate embodiment, the shell wall material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof.

In still another embodiment, the shell wall material may comprise an edible wax. Edible waxes may be derived from animals, insects, or plants. Non-limiting examples include beeswax, lanolin, bayberry wax, carnauba wax, and rice bran wax. The shell wall material may also comprise a mixture of biopolymers. As an example, the shell wall material may comprise a mixture of a polysaccharide and a fat.

In yet another embodiment, the shell wall material may comprise a semi-synthetic polymer. Semi-synthetic polymers include, but are not limited to, semi-synthetic celluloses and semi-synthetic starches. The semi-synthetic celluloses include methylcellulose, ethylcellulose, hydroxyethylcellulose, carboxymethylcellulose, hydroxypropylcellulose, hydroxypropylmethylcellulose, sulfonated cellulose, cellulose acetate, cellulose acetate phthalate, cellulose acetate trimelitate, cellulose ethyl phthalate, and viscose. Suitable semi-synthetic starches include water-soluble starch, carboxymethylated starch, dialdehyde starch, hydrophobically modified starch, oxidized starch, etherified starch, and esterified starch.

Without being bound by any particular theory, the shell wall may encapsulate the core material such that it preserves and protects the core of phospholipid-stabilized oxidizable material. The shell wall preserves the shape and integrity of the particle of phospholipid-stabilized oxidizable material. When the microcapsule is used in food products having moisture, the shell wall serves as a substantial barrier to moisture, thereby protecting and stabilizing the core of phospholipid-stabilized oxidizable material. Stated another way, the shell wall is generally substantially water impermeable. Thus, the shell wall is preferably structurally intact; that is, the shell is preferably not mechanically harmed or chemically eroded so as to permit ready entry of water into the core. Preferably, the shell is substantially water impermeable until the microparticle in a food product is ingested.

As will be appreciated by a skilled artisan, the shell wall generally is constructed such that it protects the core material during storage, but that upon ingestion, the shell wall will be compromised to permit release of the core material. Thus, the material or materials comprising the shell wall and the thickness of the shell wall can and will vary depending upon the conditions under which the microcapsule is to be utilized. That is, whether the microcapsule is added to a low moisture content food or added to a high moisture content food.

(c) Physical Properties of the Microcapsule

The size and shape of the microcapsules can and will vary without departing from the scope of the present invention. Generally, their size may be measured in terms of the diameter of a sphere that occupies the same volume as the microcapsule being measured. The characteristic diameter of a microcapsule may be directly determined, for example, by inspection of a photomicrograph. The size of the microcapsules can and will vary, depending upon the condition used to form the particles and the type of encapsulation. Typically, a microcapsule of the present invention may have a diameter from 10 nanometers to about 500 micrometers.

The size distribution of a sample of microcapsules may be measured using a particle analyzer by a laser light scattering technique. Generally, particle size analyzers are programmed to analyze particles as though they were perfect spheres and to report a volumetric diameter distribution for a sample on a volumetric basis. An example of a suitable particle analyzer is the Malvern Zeta Sizer (Malvern Instruments, Worcestershire, UK).

The thickness of a microcapsule shell wall may be an important factor in some instances. Shell walls that are too thin may have insufficient integrity to withstand mechanical forces and remain intact. Shell walls that lack mechanical integrity may be prone to defects and destruction, thereby allowing access of water to the core material. Shell walls that are too thick may be uneconomical and may delay release of the core materials in the digestive tract.

The thickness of a microcapsule shell wall of the present invention may be expressed as a percentage representing the ratio of the weight of the shell to the weight of the core material. Accordingly, the weight ratio of shell to core may be less than about 65% (e.g., between about 1% or 5% and about 65%). Alternatively, the weight ratio may be less than about 35% (e.g., between about 1% and 35%). In still another embodiment, the weight ratio is less than about 15% (e.g., between about 1% and 15%). Generally then, for microcapsules having a wall to core weight ratio between about 5% and about 15%, the equivalent thickness of shells is between about 1.5% and about 5% of the diameter of a microcapsule.

By way of example, the equivalent shell wall thickness of a microcapsule having a diameter between about 0.1 micrometers and about 60 micrometers may typically be between about 0.001 micrometers and 4 micrometers. Likewise, for microcapsule diameters between about 1 micrometers and 30 micrometers, the equivalent shell wall thickness may be between about 0.01 micrometers and 2 micrometers. For microcapsule diameters between about 1 micrometers and 6 micrometers, the equivalent shell wall thickness may typically be between about 0.01 micrometers and 0.4 micrometers.

(d) Methods of Microencapsulation

The present invention is directed toward, in part, microcapsules having a core material contained therein. Generally speaking, the core material may be encapsulated by the shell wall to form a microcapsule of the invention by methods known in the art. As will be appreciated by a skilled artisan, the encapsulation method can and will vary depending upon the compounds used to form the core material and shell wall, and the desired physical characteristics of the microcapsules themselves. Additionally, more than one encapsulation method may be employed so as to create a multi-layered microcapsule, or the same encapsulation method may be employed sequentially so as to create a multi-layered microcapsule.

Methods of microencapsulation may include spray drying, spinning disk encapsulation (also known as rotational suspension separation encapsulation), supercritical fluid encapsulation, air suspension microencapsulation, fluidized bed encapsulation, spray cooling/chilling (including matrix encapsulation), extrusion encapsulation, centrifugal extrusion, coacervation, alginate beads, liposome encapsulation, inclusion encapsulation, colloidosome encapsulation, sol-gel microencapsulation, and other methods of microencapsulation known in the art.

Methods of spray drying encapsulation are well known in the art. For instance, see S. Gouin (2004) Trends in Food Science and Technology 15:330-347 and Langrish and Fletcher (2001) Chemical Engineering Process 40:345-354. Spray drying encapsulation may include aqueous two-phase systems (Millqvist et al., (2000) J. Colloid and Interface Science 225:54-61) and multiple layered microcapsules (Edris and Benrgnstahl (2001) Nahrung/Food 45:133-37).

Methods of encapsulation utilizing the spinning disk method are known in the art (see U.S. Patent Application No. 20060078598). The spinning disk method typically uses an emulsion or suspension including the ingredient and the coating composition. The emulsion or suspension is fed to the disk surface where it can form a thin wetted layer that, as the disk rotates, breaks up into airborne droplets from surface tension forces that induce thermodynamic instabilities. The resulting encapsulated ingredients may be individually coated in a generally spherical shape or embedded in a matrix of the coating composition. Because the emulsion or suspension is not extruded through orifices, this technique permits use of a higher viscosity coating and allows higher loading of the ingredient in the coating.

Methods of microencapsulation utilizing supercritical fluids are well known in the art. For instance, see U.S. Pat. No. 6,087,003; Ribeiro et al. (2003) J. of Microencapsulation 20:97-109; Ribeiro et al. (2003) J. of Microencapsulation 20:110-128; Thies et al. (2003) J. of Microencapsulation 20:87-96; and PCT WO 1998/15348. Such methods may include Rapid Expansion of Supercritical Solutions (RESS) based methods.

Methods of encapsulation utilizing an air suspension process are well known in the art (see WO 1997/14408). Generally speaking, the core material is coated with the shell wall while suspended in an upward-moving air stream. The core materials are typically supported by a perforated plate having different patterns of holes inside and outside a cylindrical insert. The holes are generally of a size such that sufficient air is permitted to rise through the outer annular space to fluidize the settling core materials. Most of the rising air, which is generally heated, flows inside the cylinder, causing the core materials to rise rapidly. At the top, as the air stream diverges and slows, the core materials settle back onto the outer bed and move downward to repeat the cycle. Generally, the core materials pass through the inner cylinder many times in a few minutes until the encapsulation process is completed. Methods of fluidized bed encapsulation are also well known in the art. (See S. Gouin, (2004) Trends in Food Science and Technology 15:330-347 for review).

Fluidized bed encapsulation may be a top-spray, Wurster, or rotational fluidized bed encapsulation. When the core material comprises a liquid, centrifugal extrusion may be used for encapsulation. In this process, core materials comprising liquids are encapsulated using a rotating extrusion head containing concentric nozzles. A jet of core liquid is surrounded by a shell wall solution. As the jet moves through the air it breaks, owing to Rayleigh instability, into droplets of core material, each coated with the shell wall solution. While the droplets are in flight, a molten shell wall may be hardened or a solvent may be evaporated from the shell wall solution to form microcapsules.

Methods of extrusion microencapsulation are well known in the art. See Schultz (1956) Food Technology 10:57-60; U.S. Pat. No. 2,809,895; S. Gouin (2004) Trends in Food Science and Technology 15:330-347. Extrusion microencapsulation may be performed at low temperatures or high temperatures. Additionally, extrusion microencapsulation may be performed with low moisture content or high moisture content.

Methods of coacervation are well known in the art. (See S. Gouin (2004) Trends in Food Science and Technology 15:330-347 for review). As used herein, “coacervation” also refers to complex coacervation. The shell resulting after coacervation microencapsulation may or may not be cross-linked. Additionally, coacervation may be used to create multi-layered microcapsules. Such multi-layered capsules may be created solely via the coacervation process, or they may be created using a separate encapsulation process in addition to the coacervation process.

Methods of inclusion encapsulation are well known in the art. (See S. Gouin (2004) Trends in Food Science and Technology 15 330-347 for review). Generally speaking, inclusion encapsulation refers to the association of the encapsulated ingredient in a cavity-bearing shell material. The encapsulated ingredient is kept within the cavity by hydrogen bonding, Van der Waals forces, or by the entropy-driven hydrophobic effect (S. Gouin (2004) Trends in Food Science and Technology 15 pg. 340).

Methods of colloidosome encapsulation are well known in the art. (See S. Gouin (2004) Trends in Food Science and Technology 15:330-347; Dinsmore et al. (2002) Science 298:1006-1009). Typically, colloidosomes resemble liposomes, but colloidosome shells are comprises of colloid particles. The shells may by crosslinked or sintered.

Methods of encapsulation using alginate beads, liposomes, spray cooling/chilling, and sol-gel encapsulation are also well known in the art. (See S. Gouin (2004) Trends in Food Science and Technology 15:330-347 for review).

(III) Food Products

A further aspect of the present invention is the provision of a food product comprising an edible material and a microcapsule. The microcapsule comprises a core material and a shell wall that encapsulates the core material. The core material comprises a phospholipid-stabilized oxidizable material, wherein the concentration of the phospholipid in the core material ranges from about 2% to about 50% by weight of the oxidizable material. As described above in section (II)(b), the nature of the shell wall of the microcapsule will vary depending upon the type of food that the microcapsule is to be incorporated.

In one embodiment, the food product may be a liquid beverage. Non-limiting examples of a liquid beverage include milk, flavored milk drinks, goat milk, liquid yogurt, soy milk, rice milk, fruit drinks, fruit-flavored drinks, vegetable drinks, nutritional drinks, energy drinks, sports drinks, infant formula, teas, and coffee drinks.

In another embodiment, the food product may also be a dairy or an egg product. Examples of dairy products include, but are not limited to, cheese, ice cream, ice cream products, yogurt, whipping cream, sour cream, cottage cheese, buttermilk, egg whites, and egg substitutes.

In an alternate embodiment, the food product may be a cereal-based product. Non-limiting examples of food products derived from cereal include breakfast cereals, pasta, breads, baked products (i.e., cakes, pies, rolls, cookies, crackers), tortillas, granola bars, nutrition bars, and energy bars. The food product may be a nutritional supplement.

In still another embodiment, the food product may be a vegetable-derived product. Examples of vegetable-derived food products include textured vegetable proteins, tofu, corn chips, potato chips, vegetable chips, popcorn, and chocolate products.

In yet another embodiment, the food product may be a meat product or a meat analog. Examples of meat products include, but are not limited to, processed meats, comminuted meats, and whole muscle meat product. The meat may be animal meat or seafood meat. The meat analog may be a textured vegetable or dairy protein that mimics animal or seafood meat in texture. The meat analog may be part or all of the meat in a food product. The food product may also be a canned food product to which the microcapsule is added to prevent oxidation during the heating process.

In yet another embodiment, the food product may be a product for animals. The animal may be a companion animal, an agricultural animal, or an aquatic organism. Non-limiting examples of animal food products include canned pet foods, dried pet foods, agricultural animal feeds, and agricultural animal feed supplements. The feeds may be pelleted, extruded, or formed by other methods. The feeds or feed supplements may be liquid. Examples include a nursery diets for monogastric animals, calf milk replacer, or fish and other oils used to supplement animal feeds.

Another aspect of the invention provides for food products treated with the composition of the invention. The composition may be sprayed on or applied to a food product. Non-limiting examples of suitable food products include food bars, nutrition bars, snacks, nuts, oats, cookies, crackers, dried fish or seafood products, and pet foods or pet snacks. The composition may be added directly to oxidation sensitive foods. Examples include, but are not limited to, cooking oils, frying oils, spray-on oils, salad dressings, margarines, nut oils, herb or spice oils, cream liquors, shelf-stable cream products, fish oils, fish sauce, nutritional supplements containing fat soluble vitamins and oils, and pharmaceutical preparations containing oxidizable lipids or oils.

(IV) Nonfood Products

A further aspect of the invention provides nonfood products comprising phospholipid-stabilized oxidative materials or microcapsules comprising phospholipid-stabilized oxidative materials. The nonfood product may be a cosmetic, a body moisturizer, or an anti-aging cream for humans, or it may be a product to prevent pet coat oil oxidation or prevent pet odor. The nonfood product may be a fragrance product or an air freshener product. The nonfood product may be a paint or varnish. The nonfood product may be a mineral oil, a synthetic oil, or a biodiesel.

### DEFINITIONS

As used herein, the term “microcapsule” refers to a composition comprising a core material and a shell wall that surrounds or encapsulates the core material.

The term “oxidizable material,” as used herein, refers to a material comprising an oxidizable lipid. The material may be a crude mixture or a highly purified preparation.

The term “phospholipid,” as used herein, generally refers to a glycerol-containing phospholipid, such as phosphatidylcholine, phosphatidylethanolamine, phosphatidylinositol, phosphatidylserine, and diphosphatidylglycerol. Lecithin comprises a mixture of glycerophospholipids.

The term “substantially water-free,” as used herein, means that the phosopholipid-stabilized oxidizable material is greater than about 90% water-free, more preferably, greater than about 95% water-free, still more preferably greater than about 97% water-free, and even more preferably, greater than 99% water-free.

As various changes could be made in the above composition, products and methods without departing from the scope of the invention, it is intended that all matter contained in the above description and in the examples given below, shall be interpreted as illustrative and not in a limiting sense.

### EXAMPLES

The following examples illustrate various embodiments of the invention.

### Example 1

### Stability of Lecithin-Stabilized Omega-3 Fish Oil Microcapsules

The ability of lecithin to prevent the oxidation of omega-3 fish oils was examined by preparing microcapsules comprising omega-3 fish oils and lecithin. For this, emulsions of fish oil prepared with increasing concentrations of lecithin were prepared, encapsulated, and spray dried. The percentage of lecithin to fish oil ranged from 0.1% to 50% (see Table 1).

Preparation of microcapsules. Solution A was prepared by heating 4781 parts of tap water to the boiling point and then cooling it to 70-80° C. To this was added 14 parts of sodium citrate and the amounts of lecithin listed in Table 1. Two different preparations of lecithin were used: Solec 8160, an enzyme modified lecithin preparation, and Solec F, a non-modified lecithin. The mixture was maintained at 70° C. and stirred until the powders had dissolved. Then 105 parts of Supro® EX 45 soy protein isolate was added and the mixture was heated to 70-75° C. and stirred until the soy protein was dissolved. A 33% aqueous citric acid solution was added to adjust the pH to 3.7-3.8. The mixture was homogenized at 4000 pounds per square inch to obtain a good dispersion, to which omega-3 fish oil (ROPUFA, DSM Nutriceuticals, Parsippany, N.J.) was added, in the amounts listed in Table 1, and the slurry was mixed for 1-2 minutes. The slurry was subjected to a two-stage homogenization at 6500 pounds per square inch for the first stage and 500 pounds per square inch for the second stage to obtain an emulsion comprising particles of fish oil and lecithin.

Solution B was prepared by mixing 2800 parts of tap water and 800 parts of gelatin at 400° C. The pH was adjusted to 6.5 with aqueous sodium hydroxide and 400 parts of gum arabic was added to obtain the outer coating composition. The solution was maintained at 40° C., and 4000 parts of Solution A was added to the vessel containing Solution B (4000 parts). The pH of the mixture was immediately lowered to a value of 4 by the addition of a 33% aqueous citric acid solution. The mixture was then cooled to 5° C. with stirring, and then was sprayed dried using an inlet temperature of 200° C. and an outlet temperature of 100° C. The microcapsule preparations were stored at 4-5° C.

TABLE 1
Amounts of Lecithin and Fish Oil Used to Make Microcapsules

| Percentage of | | | |
|---|---|---|---|
| Lecithin | Solec F Lecithin | Solec 8160 Lecithin | Fish Oil |
| to Fish Oil | (parts) | (parts) | (parts) |
| 0.1 | 1.1 | 1.1 | 2098 |
| 0.5 | 5.5 | 5.5 | 2089 |
| 1 | 11 | 11 | 2078 |
| 3 | 31 | 31 | 2034 |
| 6.4 | 63 | 63 | 1974 |
| 10 | 95.5 | 95.5 | 1909 |
| 20 | 175 | 175 | 1750 |
| 30 | 242.3 | 242.3 | 1615 |
| 40 | 300 | 300 | 1500 |
| 50 | 350 | 350 | 1400 |

Oxidative stability. The oxidation stability of the microcapsules prepared above was evaluated using the Oxidation Stability Index (OSI) method, a method approved by the American Oil Chemists Society (AOCS Official Method Cd 12b-92). This method measures the period of time during which oils are resistant to oxidation. After this period of time, or the induction period, the rate of oxidation accelerates rapidly. During the OSI procedure, a stream of air is passed through an oil sample, which is heated to 110° C., and the effluent air from the oil sample is bubbled through a test vessel containing deionized water, whose conductivity is continuously monitored over time. As the oil oxidizes, volatile organic acids are generated and become trapped in the water, thereby increasing its conductivity. The OSI value is defined as the induction period in hours and mathematically represents the inflection point (second derivative) of the conductivity curve that reflects the maximum change in the oxidation rate. The higher the OSI value, the more stable the oil.

A sample of each of the microcapsules was mixed with an equal weight of inert mineral oil. Baseline samples comprising omega-3 fish oil (baseline A) and a 1:1 mix of Solec 8160 and Solec F lecithins (baseline B) were also run. The OSI values are presented in Table 2. Lecithin stabilized the fish oil in the core of the microcapsules in a concentration dependent manner.

TABLE 2
Stability of Lecithin Stabilized Oil Microcapsules

| | Percentage of lecithin | OSI Value |
|---|---|---|
| Sample | to oil | (hr) |
| Baseline A | 0 | 1.75 |
| Baseline B | 0 | 1.1 |
| Microcapsule | 0.1 | 5.75 |
| Microcapsule | 0.5 | 4.22 |
| Microcapsule | 1 | 5.45 |
| Microcapsule | 3 | 13.3 |
| Microcapsule | 6.4 | 23.0 |
| Microcapsule | 10 | 36.7 |
| Microcapsule | 20 | 62.2 |
| Microcapsule | 30 | 67.1 |
| Microcapsule | 40 | 72.4 |
| Microcapsule | 50 | 83.6 |

### Example 2

### Stability of Encapsulated versus Non-encapsulated Lecithin-Stabilized Oils

The stability of encapsulated and non-encapsulated preparations of lecithin-stabilized oils was compared. Microcapsules comprising omega-3 fish oil and different percentages of lecithin ranging from 0.1% to 50% (by weight of the oil) were prepared and encapsulated as essentially described in Example 1. Lecithin-stabilized fish oils were prepared by dissolving the appropriate amount of lecithin (3% to 30%) in water (with or without an added protein), adding the appropriate volume of omega-3 fish oil, and homogenizing the mixture to create an emulsion. The water was removed from the emulsion by spray drying to form the lecithin-stabilized oils.

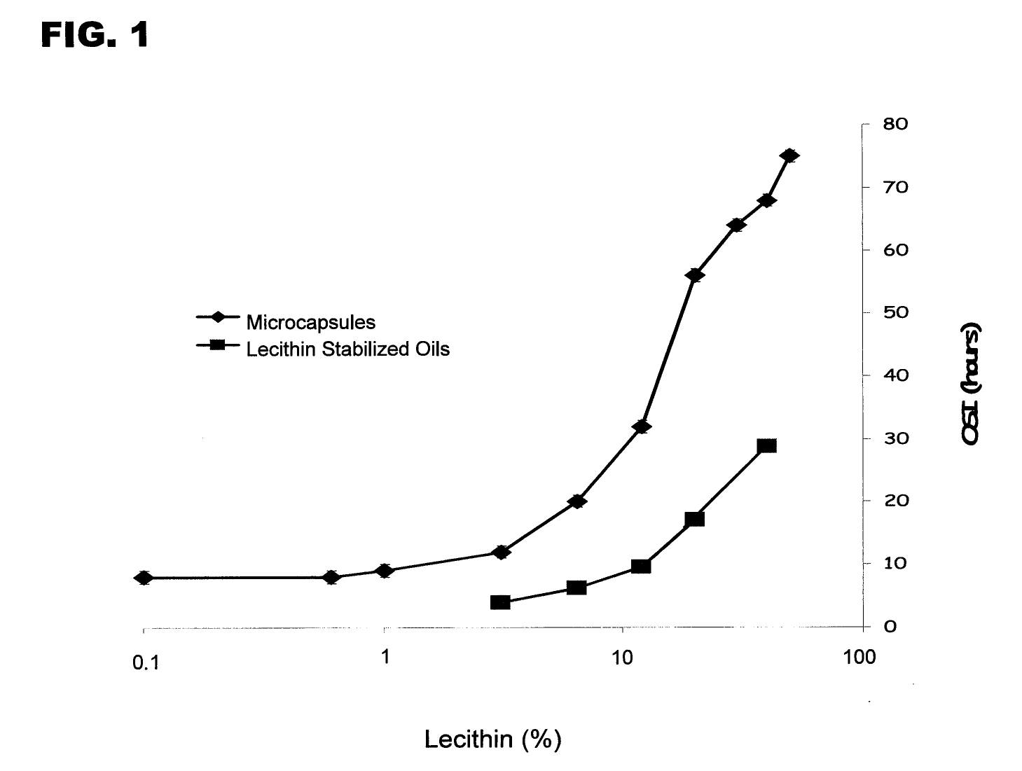

The oxidative stability of the lecithin-stabilized oils and the microcapsules were measured using the OSI method essentially as described in Example 1. As shown in FIG. 1, the microcapsules had higher OSI values, i.e., were more stable, than the lecithin-stabilized oils at every level of lecithin.

### Example 3

### Peroxide Values of Lecithin-Stabilized Oils

The oxidative stability of lecithin-stabilized fish oils was also analyzed by directly measuring the levels of peroxides in the preparations. The peroxide values (PV) are expressed as mmol/kg of oil. Lecithin-stabilized omega-3 fish oils comprising 3.1%, 6.4%, 12%, 20%, or 40% of lecithin (by weight of the oil) were prepared as described in Example 2 and were stored at 4°-5° C. Peroxide values were determined in the lecithin-stabilized oils on days 0, 3, 6, 9, 16, and 24.

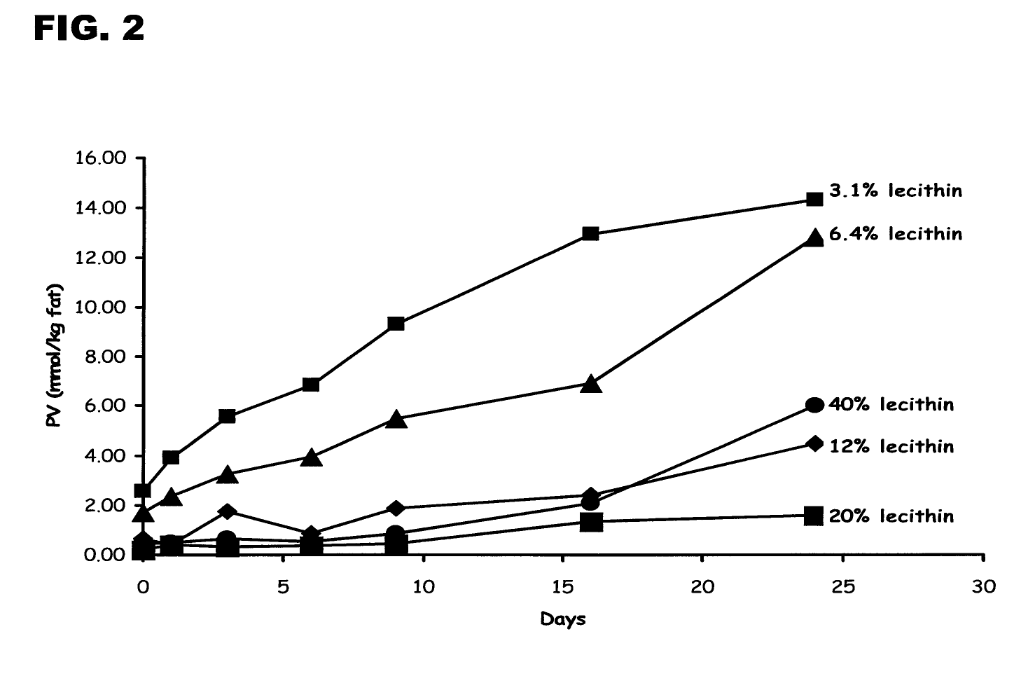

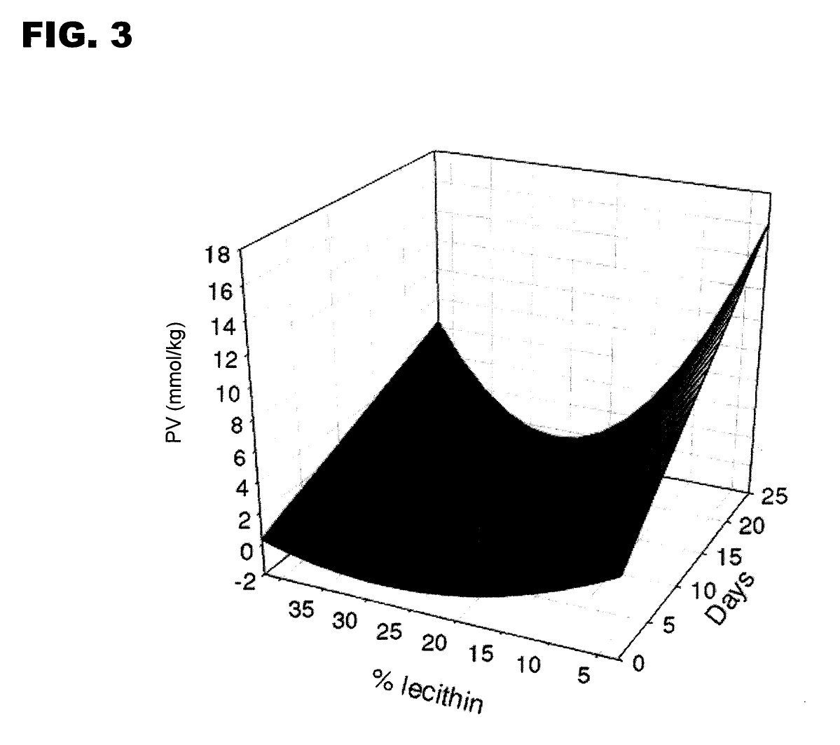

As shown in FIG. 2, the best protection was provided by 20% lecithin, at every time point. Lower and higher percentages of lecithin provided less oxidative stability. The quadratic plot presented in FIG. 3 confirms that the optimal stabilization occurred at a lecithin concentration of about 25-30%, with lower and higher concentrations of lecithin providing less stabilization. Furthermore, this biphasic effect was more marked over time.

### Example 4

### Stability of Microcapsules as Monitored by Propanal Production

Propanal is the aldehyde of the 3-carbon propyl group and serves as an excellent marker for the oxidation of omega-3 fatty acids. Thus, the production of propanal can be used to deduce that amount of oxidative degradation of omega-3 oils and determine the subsequent stabilization provided by lecithins. Lecithin itself contains unsaturated fatty acids, especially the omega-6 fatty acid, linoleic acid (18:2), whose concentration is greater than 50%. A marker for the oxidative breakdown of linoleic aid is hexanal, the aldehyde of the 6-carbon hexanyl group. Gas chromatography-flame ionization detection (GC-FID) methods were optimized to detect propanal and hexanal.

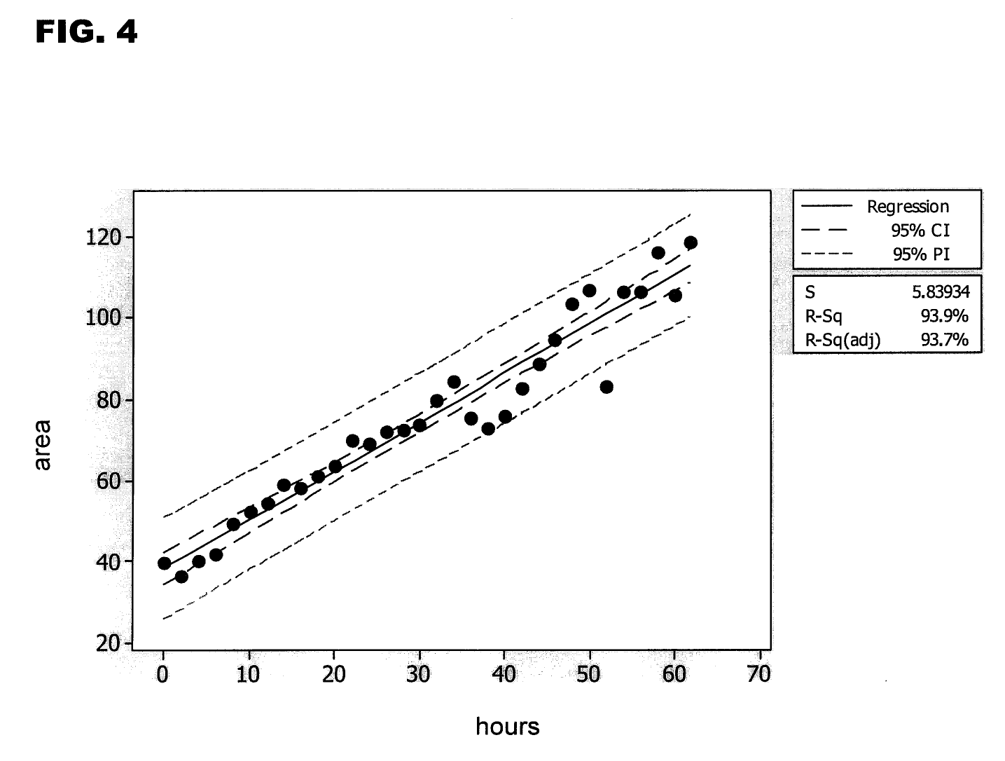

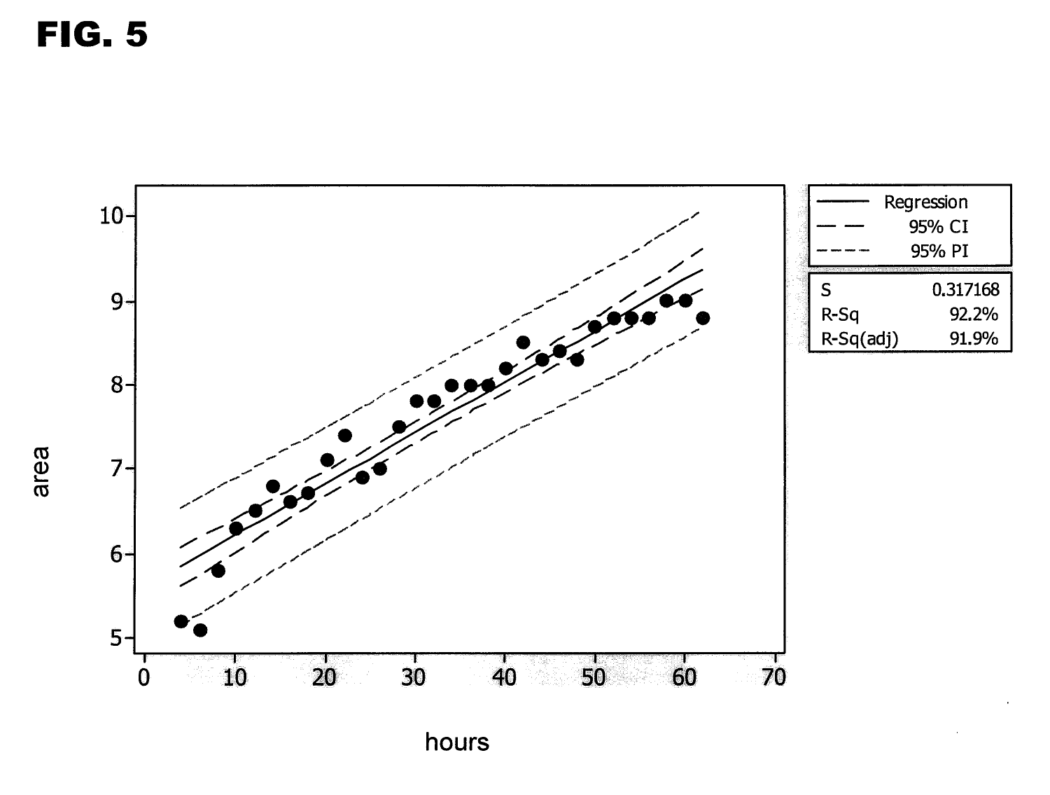

Microcapsules comprising lecithin-stabilized omega-3 fish oil were prepared essentially as described in Example 1. The concentration of the lecithin in the core material of the microcapsule was 0.1%, 6.4%, 12%, 30%, or 40% by weight of the fish oil. The levels of propanal were measured at days 0, 1, 2, and 3. Of the percentages of lecithin tested, the lowest levels of propane were observed in microcapsules comprising 12% lecithin (data not shown). Propanal development was monitored in microcapsules comprising 12% lecithin over a period of about 60 hours. The peak areas under the curves are presented in FIG. 4. This experiment revealed that the production of propanal was linear over time. The development of hexanal, which is breakdown product of omega-6 acids in lecithin, was also monitored in microcapsules comprising 12% lecithin over time (FIG. 5). The production of hexanal was also linear over time, but the levels of hexanal were an order of magnitude lower than those of propanal.

### Example 5

### Structure of a Microcapsule

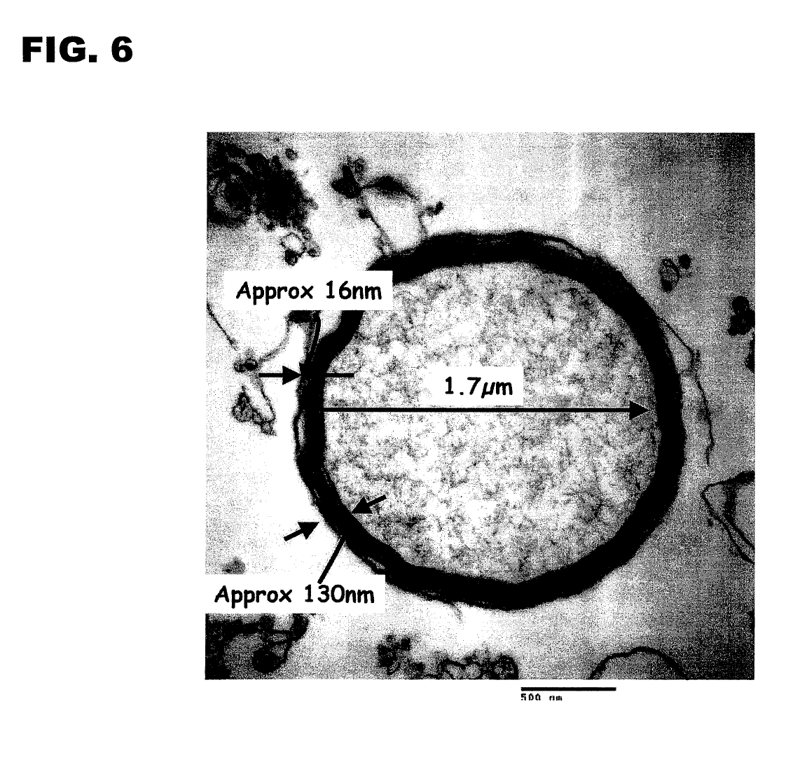

Microcapsules comprising omega-3 fish oil, 6.4% lecithin, and soy protein were prepared essentially as described in Example 1. Microcapsules were prepared for TEM by dehydration in ethanol and propylene oxide, after which they were embedded in Epon resin. Ultra thin sections (˜50 nm) were cut using an ultra-microtome, positioned on a TEM grid, and viewed via TEM. FIG. 6 presents an image of a typical microcapsule. The diameter of the core material was about 1.7 μm and the shell wall had a thickness of about 130 nm. Note, that the shell wall comprises many thinner layers of about 16 nm.

### Example 6

### Additional Antioxidants Further Stabilize the Microcapsules

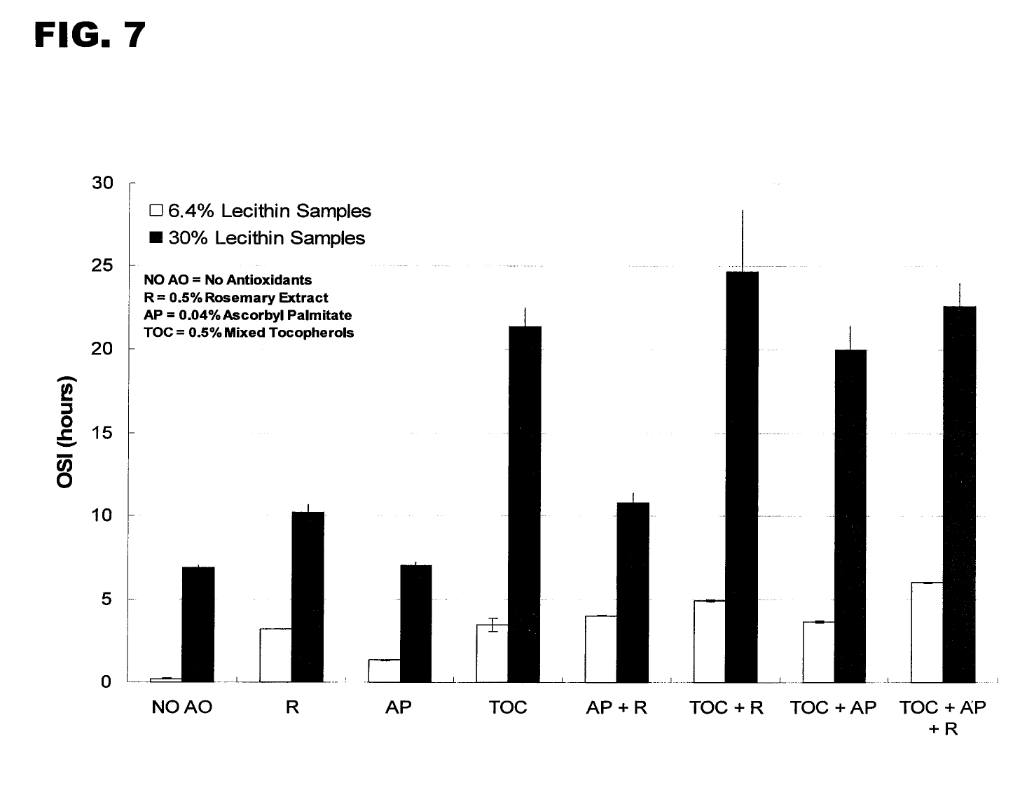

Microcapsules comprising omega-3 fish oil and either 6.4% or 30% lecithin (by weight of the oil) were prepared alone or with 0.5% of rosemary extract, 0.04% of ascorbyl palmitate, 0.5% of mixed tocopherols, or a combination thereof, essentially as described in Example 1. The oxidative stability of these preparations was evaluated using the OSI method, which are plotted in FIG. 7. Microcapsules comprising 30% lecithin were stabilized longer than those comprising 6.4% lecithin. While the addition of most antioxidants increased the stability of the microcapsules somewhat, the addition of the mixed tocopherols produced the greatest protective effect in the microcapsules comprising 30% lecithin.

### Example 7

### Analysis of Volatiles in the Lecithin-Stabilized Oils

Although the addition of omega-3 fatty acids provides health benefits, the addition of fish oils to food products raises the possibility that the food will taste and/or smell fishy. To address this possibility, the levels of five volatiles that are presumed to be responsible for fish odor/flavor were measured in 6% lecithin-stabilized oil and 23% lecithin-stabilized oil. A gas chromatography mass spectrometry (GC MS) method was optimized to measure 1-penten-3-one, E-2-hexenal, Z-4-heptenal, E,E-2,4-heptadienal, and E,Z-2,6-nonadienal.

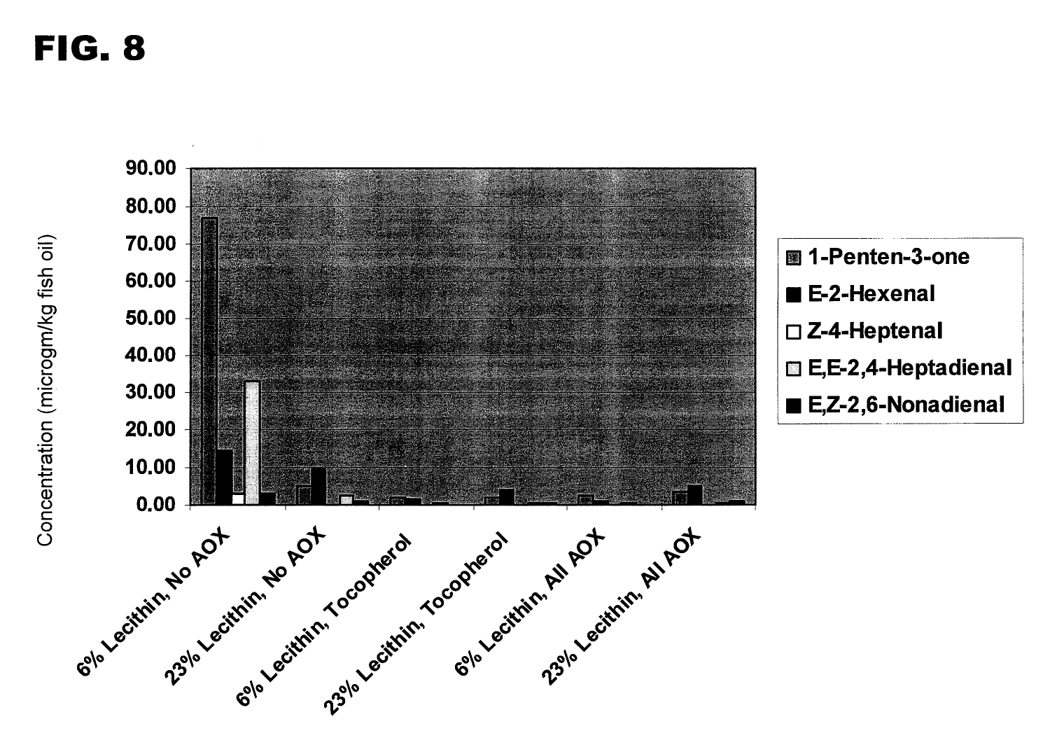

As shown in FIG. 8, the levels of the five volatiles were lower in the 23% lecithin-stabilized oil without additional antioxidants than in the 6% lecithin-stabilized oil without additional antioxidants. The addition of tocopherols drastically decreased the levels of these volatiles in both preparations. The further addition of rosemary extract and ascorbyl palmitate along with the mixed tocopherols did not provide any further reduction in the levels of these compounds.

### Example 8

### Sensory Analysis of the Microcapsules

A proprietary sensory screening method, the Solae Qualitative Screening (SQS) method, was used to assess the degree of “fishy” flavor in the microcapsules as compared to a control sample. Microcapsules comprising omega-3 fish oil and differ percentages of lecithin (1% to 30%) were prepared and provided to a panel of tasters. The control sample was a commercial fish oil (Ocean Nature Meg-3 encapsulated fish oil). To rate each test sample, each assessor swirled each cup three times, keeping the bottom of the cup on the table. After the sample sat for 2 seconds, each assessor sipped about 10 ml (2 tsp), swished it about his/her mouth for 10 seconds, and then expectorated. The assessor then rated the differences between the test sample and the control sample according to the scale presented in Table 3. The less “fishy” the test sample, the lower the score.

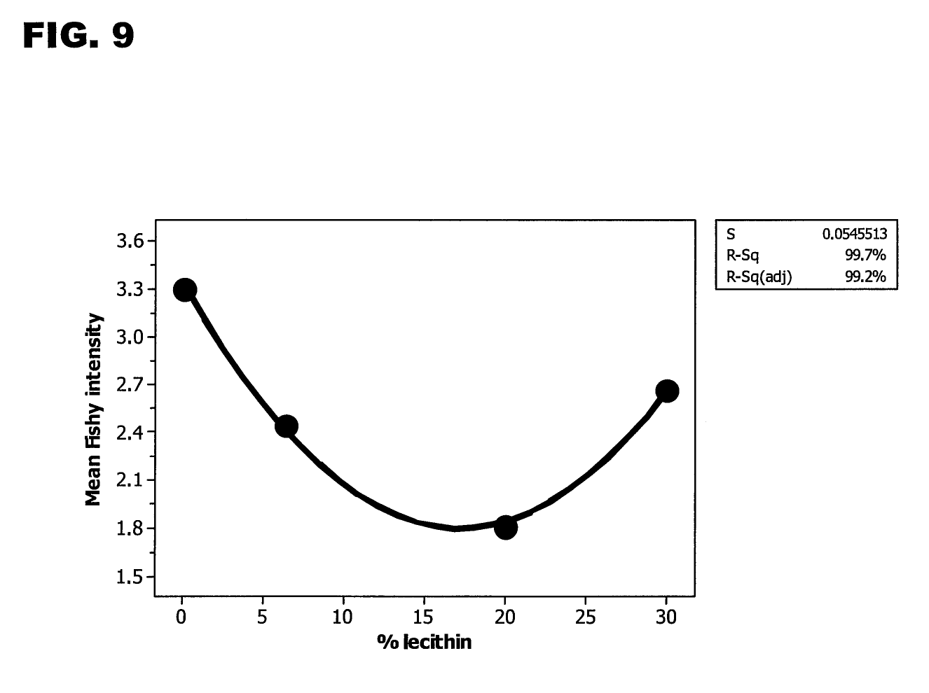

The mean score of fishy flavor for each concentration of lecithin is presented in FIG. 9. The lowest SQS scores were obtained with microcapsules comprising 20% lecithin. These data support the chemical data presented above.

TABLE 3
SQS Scoring System

| SQS | | |
|---|---|---|
| Score | Scale | Definition |
| 5 | Match | The test sample has virtually identical |
| | | sensory characteristics to the control sample |
| | | by appearance, aroma, flavor and texture. |
| 4 | Slight | The test sample has one or multiple |
| | differ- | ‘slight’ differences from the |
| | ence | control sample. These differences might |
| | | not be noticed if not in a side-by-side |
| | | comparison with the control. |
| 3 | Moderate | The test sample has one or multiple |
| | differ- | ‘moderate’ differences from the |
| | ence | control sample. These differences would be |
| | | noticeable in a side-by-side comparison |
| | | of the two samples after one tasting of each. |
| 2 | Extreme | The test sample has one or multiple |
| | differ- | ‘extreme’ differences from the |
| | ence | control sample. These differences would be |
| | | noticed even if not in a side-by-side |
| | | comparison. |
| 1 | Reject | The test sample has obvious defects |
| | | that make it different from the |
| | | control sample. |

### Example 9

### SQS Scores of Chocolate Flavored Food Bars

The sensory characteristics of the microspheres were further characterized by preparing chocolate flavored food bars with 100 mg of microcapsules comprising either 6.4% or 30% lecithin. The SQS analysis used above was expanded to include additional sensory attributes (see Table 4). The overall taste, chocolate flavor, and grainy mouth feel of the samples were also evaluated. The SQS analysis was further modified to assess directional quantitative differences between the test sample and the control sample. If a test sample was rated a 2, 3, or 4, then the rating was expanded to allow the taster to rate the test sample as having “more” or “less” of the attribute relative to the control sample (which was assigned a 0). Thus, if the test sample had slightly more, moderately more, or extremely more of the attribute than the control sample, then scores of +1, +2, +3, respectively, were assigned. Likewise, if the test sample had slightly less, moderately less, or extremely less of the attribute than the control sample, then scores of −1, −2, −3, respectively, were assigned.

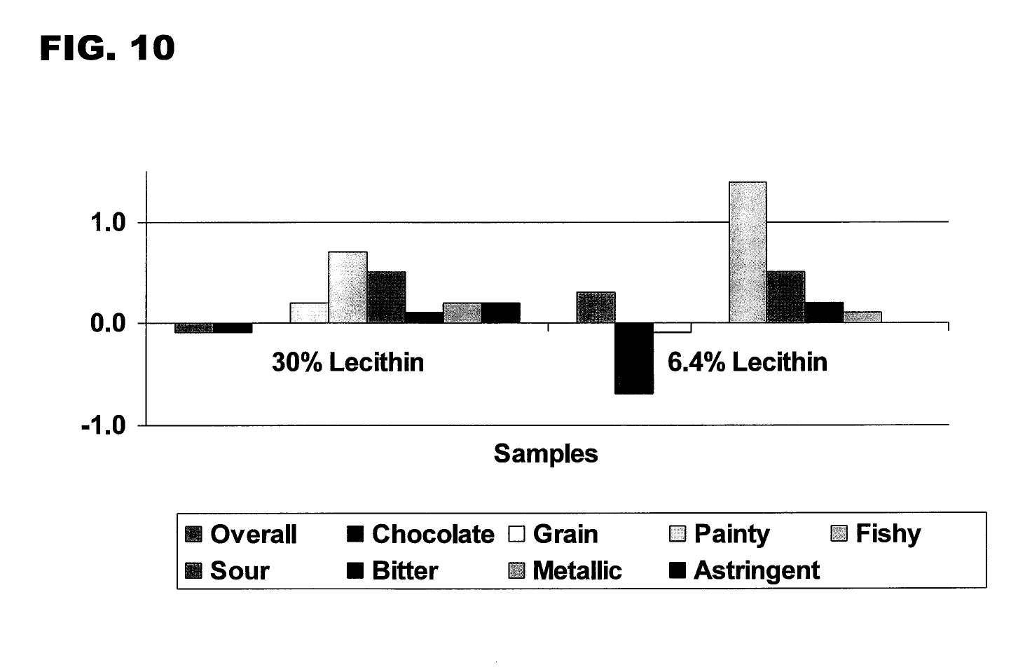

The diagnostic scores, which reflect the differences between the test samples and the control sample, are plotted in FIG. 10. In general, the bars comprising 30% lecithin microcapsules had a less fishy taste and a greater chocolate taste than those comprising 6.4% lecithin microcapsules.

TABLE 4
Sensory Attributes

| Attribute | Definition | Reference |
|---|---|---|
| Painty | Aromatic associated with moderately | Linseed oil |
| | oxidized oils, similar to linseed | |
| | oil or oil-based paints. | |
| Fishy | Aromatic associated with trimethyl- | Cod liver oil |
| | amine and old fish. | |
| Sour | The taste on the tongue stimulated | Citric acid |
| | by acid, such as citric, malic, | solution |
| | phosphoric, etc. | |
| Bitter | The taste on the tongue associated | Caffeine solution |
| | with caffeine and other bitter | |
| | substances, such as quinine and hop | |
| | bitters. | |
| Metallic | The aromatic associated with metals, | Iron tablet, canned |
| | tin or iron. | tomato juice |
| Astringent | The chemical feeling factor | Alum solution |
| | described as drying or puckering | |
| | of the oral mucosa due to tannins | |
| | or alum. | |

What is claimed is:

18. A microcapsule, the microcapsule comprising:
(a) a core material comprising an oxidizable material and a phospholipid, the concentration of the phospholipid in the core material being from about 2% to about 50% by weight of the oxidizable material; and
(b) a shell wall that encapsulates the core material.

2. The microcapsule of claim 1, wherein the oxidation of the oxidizable material is determined by the peroxide value (PV) method.

3. The microcapsule of claim 1, wherein the oxidizable material is a substantially unsaturated fat or substantially unsaturated oil.

4. The microcapsule of claim 1, wherein the oxidizable material is an oxidizable oil selected from the group consisting of fish oil, marine oil, vegetable oil, and algal oil.

5. The microcapsule of claim 1, wherein the oxidizable material is selected from the group consisting of an omega-3 fatty acid, an omega-6 fatty acid, and an omega-9 fatty acid.

6. The microcapsule of claim 1, wherein the phospholipid is selected from the group consisting of phosphatidylcholine, phosphatidylethanolamine, phosphatidylinositol, and phosphatidylserine.

7. The microcapsule of claim 1, wherein the phospholipid is a lecithin.

8. The microcapsule of claim 1, wherein the concentration of the phospholipid in the core material is from about 15% to about 35% by weight of the oxidizable material.

9. The microcapsule of claim 1, wherein the concentration of the phospholipid in the core material is from about 25% to about 30% by weight of the oxidizable material.

10. The microcapsule of claim 1, wherein the core material further comprises a protein selected from the group consisting of a vegetable protein, an animal protein, a fungal protein, and a microbial protein.

11. The microcapsule of claim 10, wherein the protein is selected from the group consisting of soy protein, corn protein, pea protein, wheat protein, casein, whey protein, and gelatin.

12. The microcapsule of claim 1, wherein the core material further comprises an antioxidant other than a phospholipid selected from the group consisting of tocopherols, ascorbyl palmitate, and rosemary extract.

13. The microcapsule of claim 1, wherein the shell wall is selected from the group consisting of gelatin, gum arabic, and a high temperature melting fat or oil.

14. The microcapsule of claim 1, wherein the shell wall is substantially water impermeable.

15. The microcapsule of claim 1, wherein the oxidizable material is an omega-3 fatty acid, the phospholipid is a lecithin, and the shell wall is substantially water impermeable.

16. The microcapsule of claim 15, wherein the concentration of the lecithin in the core material is from about 15% to about 35% by weight of the oxidizable material.

17. A food product, the food product comprising:
(a) an edible material; and
(b) a microcapsule, the microcapsule comprising a core material and a shell wall that encapsulates the core material, the core material comprising an oxidizable material and a phospholipid, the concentration of the phospholipid in the core material being from about 2% to about 50% by weight of the oxidizable material.

19. The food product of claim 17, wherein the edible material is a liquid beverage.

20. The food product of claim 17, wherein the edible material is selected from the group consisting of a dairy product, a cereal-based product, a bakery product, a food bar, a vegetable-derived product, a meat product, a meat analog product, and a nutritional supplement.

21. The food product of claim 17, wherein the oxidizable material is an omega-3 fatty acid and the phospholipid is a lecithin.

22. The food product of claim 20, wherein the concentration of lecithin in the core material is from about 25% to about 30% by weight of the oxidizable material.

23. A method for reducing the oxidation of an oxidizable material, the method comprising contacting the oxidizable material with a phospholipid in a substantially water-free environment, wherein the percentage of phospholipid is from about 2% to about 50% by weight of the oxidizable material.

24. The method of claim 22, wherein the oxidation of the oxidizable material is determined by the peroxide value (PV) method.

25. The method of claim 22, wherein the oxidizable material is a substantially unsaturated fat or substantially unsaturated oil.

26. The method of claim 22, wherein the oxidizable material is an oxidizable oil selected from the group consisting of fish oil, marine oil, vegetable oil, and algal oil.

27. The method of claim 22, wherein the oxidizable material is polyunsaturated fatty acid selected from the group consisting of an omega-3 fatty acid, an omega-6 fatty acid, and an omega-9 fatty acid omega fatty acid.

28. The method of claim 22, wherein the phospholipid is selected from the group consisting of phosphatidylcholine, phosphatidylethanolamine, phosphatidylinositol, and phosphatidylserine.

29. The method of claim 22, wherein the phospholipid is a lecithin.

30. The method of claim 22, wherein the oxidizable material is an omega-3 fatty acid and the phospholipid is a lecithin.
